(19) United States
(12) Patent Application Publication
(10) Pub. No.: US 2026/0023046 A1
(43) Pub. Date: Jan. 22, 2026

(54) FUNCTIONALIZED FABRIC ELECTRODE FOR ELECTROCHEMICAL DETECTION OF RESIDUAL CORROSION INHIBITORS IN WATER SYSTEM

(71) Applicants: Saudi Arabian Oil Company, Dhahran (SA); King Fahd University of Petroleum and Minerals, Dhahran (SA)

(72) Inventors: Abdullatif I. Abdulhadi, Dhahran (SA); Nayef M. Alanazi, Dhahran (SA); Nayif Al-Rasheedi, Dammam (SA); Sulayman Oladepo, Edmonton (CA); Basheer Chanbasha, Karimangalam (IN)

(73) Assignees: Saudi Arabian Oil Company, Dhahran (SA); King Fahd University of Petroleum and Minerals, Dhahran (SA)

(21) Appl. No.: 18/780,128

(22) Filed: Jul. 22, 2024

(51) Int. Cl. G01N 27/48; G01N 27/30; G01N 33/18

(52) U.S. Cl. CPC G01N 27/48; G01N 27/30; G01N 33/18

(57) ABSTRACT

A method for electrochemical detection of residual corrosion inhibitor in a water system is provided. In the method, a functionalized fabric sensor is provided having a textile fabric with a surface coating. The textile fabric is functionalized by the metal particles to form a working electrode. A water sample suspected of comprising a nitrogen-based corrosion inhibitor is collected, and the water sample is then added to an electrolyte solution in an electrochemical cell. The presence of the corrosion inhibitor is then detected in the water sample in the electrolyte solution of the electrochemical cell, where the electrochemical cell includes the functionalized fabric sensor as the working electrode, a reference electrode, and a counter electrode.

### TECHNICAL FIELD

The present disclosure relates to methods for detecting corrosion inhibitors, and in particular methods for electrochemical detection of corrosion inhibitors in water samples.

### BACKGROUND

Monitoring of levels of residual corrosion inhibitors in water systems can provide an added assurance of protection against corrosion, even in the presence of multiple expensive monitoring techniques such as coupons, or electrochemical probes. Corrosion monitoring, however, is often considered to be of limited value in areas where the most severe corrosion occurs, such as in inaccessible low spot areas of water systems.

One option to ensure additional water system protection from corrosion is the measurement of corrosion inhibitor concentration in water. Specifically, because the effectiveness of these inhibitors after injection may decrease, thereby leaving metal surfaces vulnerable to corrosion, it is important to be able to detect the residual presence of corrosion inhibitors or measure their effectiveness to ensure that the metal surfaces remains protected as part of a robust corrosion inhibitor management program.

Many water supply wells in the oil and gas industry that are used to maintain reservoir pressure are protected by squeezing corrosion inhibitors into the wells, while wash water wells that have been used to reduce the salts in crude oil in gas-oil separation plants (GOSP) are treated with surface injections. However there is a lack of reliable technologies to detect the residual of corrosion inhibitors in these water systems. Conventional management programs have set fixed intervals for squeezing corrosion inhibitors into the water supply wells to ensure adequate corrosion inhibitor levels, but this strategy can negatively impact the integrity of the downhole tubing and surface piping. Additionally, the lack of adequate surface corrosion monitoring tools increase the risk of leakage and the risk of disturbing the crude supply.

Conventional techniques for corrosion inhibitor monitoring in water systems have generally involved sophisticated chromatography and spectroscopy methods. However, these methods can be costly, time intensive, and have limited portability, which can limit the ability to deploy them in the field.

In regard to the above background information, the present disclosure is directed to provide a technical solution for detection and measurement of corrosion inhibitors in water systems.

### SUMMARY

In a first aspect, a method for electrochemical detection of residual corrosion inhibitor in a water system is provided. In the method, a functionalized fabric sensor is provided, wherein the functionalized fabric sensor comprises a textile fabric having a surface coating comprising metal particles, and wherein the textile fabric is functionalized by the metal particles to form a working electrode. A water sample suspected of comprising a corrosion inhibitor is then collected, wherein the corrosion inhibitor is a nitrogen-based corrosion inhibitor. The water sample is then added to an electrolyte solution in an electrochemical cell. The presence of the corrosion inhibitor is then detected in the water sample in the electrolyte solution of the electrochemical cell. The electrochemical cell includes the functionalized fabric sensor as the working electrode, a reference electrode, and a counter electrode, and the electrodes are operatively connected to a potentiostat, and the electrochemical cell is utilized at a predetermined potential to produce a measurable current signal to detect the presence of the corrosion inhibitor.

In another aspect, the metal particles comprise silver nanoparticles, and the silver nanoparticles modify the working electrode to improve conductivity and enhance detection of the corrosion inhibitor.

In another aspect, the surface coating comprises a sol-gel solution.

In another aspect, the electrochemical cell utilizes a cyclic voltammetry technique or a linear sweep voltammetry technique.

In another aspect, the corrosion inhibitor is a nitrogen-based corrosion inhibitor. In a further aspect, the nitrogen-based corrosion inhibitor is a quaternary amine, an imidazoline, an amide, or combinations thereof.

In another aspect, the electrolyte solution comprises a potassium compound. In a further aspect, the potassium compound is KCl or K₃Fe(CN)₆ (potassium hexacyanoferrate (III)).

In another aspect, the electrolyte solution further comprises a buffer solution. In a further aspect, the buffer solution comprises phosphate-buffered saline (PBS).

In another aspect, a concentration of the corrosion inhibitor in the water sample is determined based on voltammetric measurements of the system.

In another aspect, the textile fabric is a non-woven textile, cotton, polyester, or a blended textile.

In another aspect, the textile fabric is precleaned.

In another aspect, the corrosion inhibitor has a concentration in the water sample in the range of approximately 0.01-0.075%.

In another aspect, the water sample is added to the electrolyte solution at a ratio of 1 part water sample to 9 parts electrolyte solution.

Any combinations of the various embodiments and implementations disclosed herein can be used. These and other aspects and features can be appreciated from the following description of certain embodiments and the accompanying drawings and claims.

### BRIEF DESCRIPTION OF THE DRAWINGS

The processes of the present disclosure will be described in more detail below and with reference to the attached drawings.

FIGS. 1A-1B display diagrams of exemplary voltammetry system configurations in accordance with one or more embodiments.

FIG. 2A displays a diagram of an exemplary method for functionalizing a fabric electrode in accordance with one or more embodiments.

FIG. 2B displays energy dispersive x-ray spectroscopy (EDX) images of a cotton fabric functionalized with Ag nanoparticles with elemental distribution (top image), and a regular scanning electron microscopy (SEM) image of the cotton fabric functionalized with Ag nanoparticles in accordance with one or more embodiments.

FIG. 2C displays energy dispersive x-ray spectrometry (EDX) elemental map of Ag, C, and O, in the cotton fabric electrode in accordance with one or more embodiments.

FIG. 3 displays an SEM image (left panel) and EDX elemental image (right panel) of a functionalized silver fabric electrode in accordance with one or more embodiments.

FIG. 4 displays an EDX elemental map of a functionalized silver fabric electrode in accordance with one or more embodiments.

FIG. 5 displays a flow diagram of an exemplary method for electrochemical detection of residual corrosion inhibitor in a water system in accordance with one or more embodiments.

FIGS. 6A-6B display cyclic voltammetry (CV) results for various functionalized working electrodes (6A: fabric electrode, platinum electrode; 6B: glassy carbon, nickel foam) in 0.5 mM solution of K₃Fe(CN)₆ for determining electrochemical activity of the functionalized electrodes in accordance with one or more embodiments.

FIG. 7 displays CV results for a functionalized fabric electrode, in 0.5 mM solution of K₃Fe(CN)₆ for determining electrochemical activity of the functionalized fabric electrode in accordance with one or more embodiments.

FIG. 8 displays CV results of various concentrations (0.01%, 0.025%, 0.050%) of a first commercial corrosion inhibitor in water with the fabric electrode in the presence of a KCl supporting electrolyte and PBS buffer at pH 7 at room temperature in accordance with one or more embodiments.

FIG. 9 displays CV results of various concentrations (0.025%, 0.050%, 0.075%) of a second commercial corrosion inhibitor in water with the fabric electrode in the presence of a KCl supporting electrolyte and PBS buffer at pH 7 at room temperature in accordance with one or more embodiments.

FIG. 10 shows a linear calibration curve for the first commercial inhibitor sample in the concentration range of 0.0 to 0.05% in accordance with one or more embodiments.

FIG. 11 shows a linear calibration curve for the second commercial inhibitor sample in the concentration range of 0.0 to 0.075% in accordance with one or more embodiments.

FIGS. 12-14 display the cyclic voltammograms for three water treated samples, sample 1 (FIG. 12), sample 2 (FIG. 13), and sample 3 (FIG. 14), collected and analyzed using the developed fabric electrode and CV technique where the samples were analyzed at 10 times dilution in accordance with one or more embodiments.

### DETAILED DESCRIPTION

Disclosed herein are methods for electrochemical detection of residual corrosion inhibitor in a water system, such as treated water systems in the oil and gas industry. In accordance with one or more embodiments, the present methods utilize a functionalized fabric electrode to detect and measure the presence of corrosion inhibitors in a sample from a water system. More specifically, the present methods can use the functionalized fabric electrode of the present application with electrochemical voltammetry techniques (e.g., cyclic voltammetry, linear sweep voltammetry) to produce a measurable current signal in the water sample (at a given potential), thereby allowing one to detect the presence of one or more corrosion inhibitors in the water sample, as well as determine the concentration of the corrosion inhibitors in the sample. In other words, in one or more embodiments, the present methods uses functionalized fabric sensor as a working electrode in an electrochemical cell that helps to re-concentrate the dissolved corrosion inhibitor in the water sample at a given potential to a measurable current signal using electrochemical voltammetry techniques.

In one or more embodiments, the present methods allow for detection of corrosion inhibitors that are present at very low concentrations in the water samples (i.e., residual amounts of corrosion inhibitors). For example, in one or more embodiments, the present methods can detect corrosion inhibitors in a water sample when the corrosion inhibitor has a concentration in the water sample as low as approximately 0.01-0.075%. Previously, in conventional methods, it has been very challenging to detect corrosion inhibitors at low concentrations in water systems associated with oilfields due to the complexity of oilfield samples. Additionally, there has not been an international standard analytical method available to monitor residuals of corrosion inhibitors as a tool to validate their presence or availability in water systems. The present methods allow for rapid measurement of residual corrosion inhibitors in water systems (e.g., supply wells, wash water), which can replace more lengthy conventional methods that have low repeatability. Additionally, the functionalized fabric sensors of the present application are more sensitive to detection of low concentrations of corrosion inhibitors than past methods. Specifically, the functionalized fabric material of the present application helps in re-concentrating the dissolved active ingredient of the corrosion inhibitor in water systems and at a given potential to produce a measurable current signal. The electrode of the sensor can be specific for the corrosion inhibitor, which gives measurable signals at specific potential. If any other sample component gives a measurable signal, it will be at a different potential, which is clearly different from that of the target corrosion inhibitor.

These and other aspects of the present systems and methods are described in further detail below. Further, as used in the present application, the term “approximately” when used in conjunction with a numerical value refers to any number within about 5, 3 or 1% of the referenced numerical value, including the referenced numerical value.

As mentioned above, in accordance with one or more embodiments, the present application discloses methods for electrochemical detection of corrosion inhibitors (e.g., residuals of corrosion inhibitors) in a water system. In one or more embodiments of the present methods, the corrosion inhibitors that are detected in the water samples are nitrogen-based corrosion inhibitors, such as amines, imidazolines, or amides. For example, in one or more embodiments, the nitrogen-based corrosion inhibitors can comprise benzimidazole, imidazoline amides, imidazoline amino amides, and their derivatives.

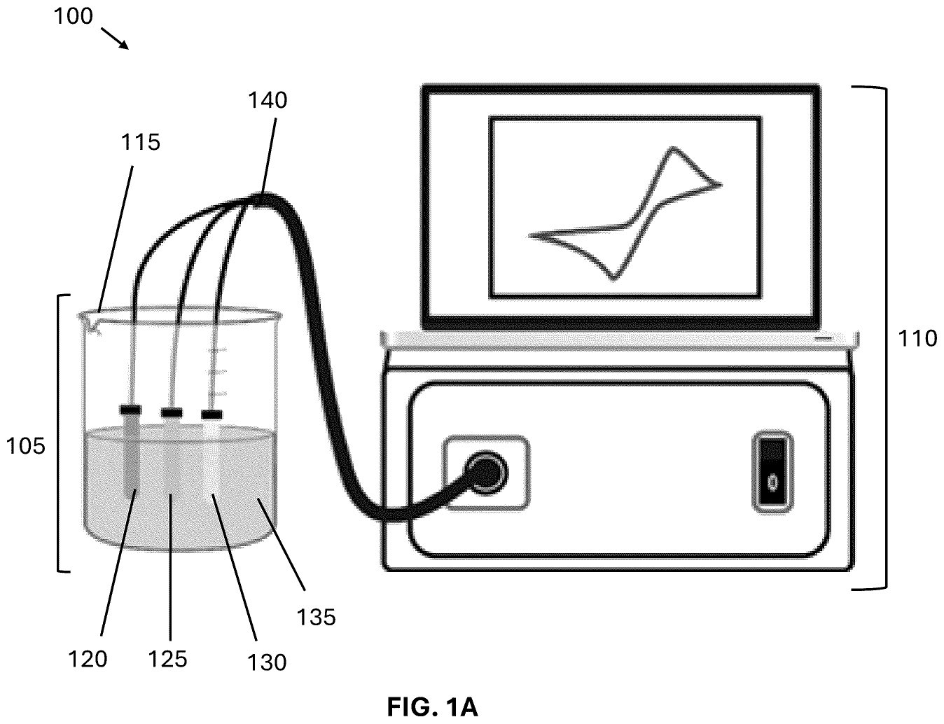

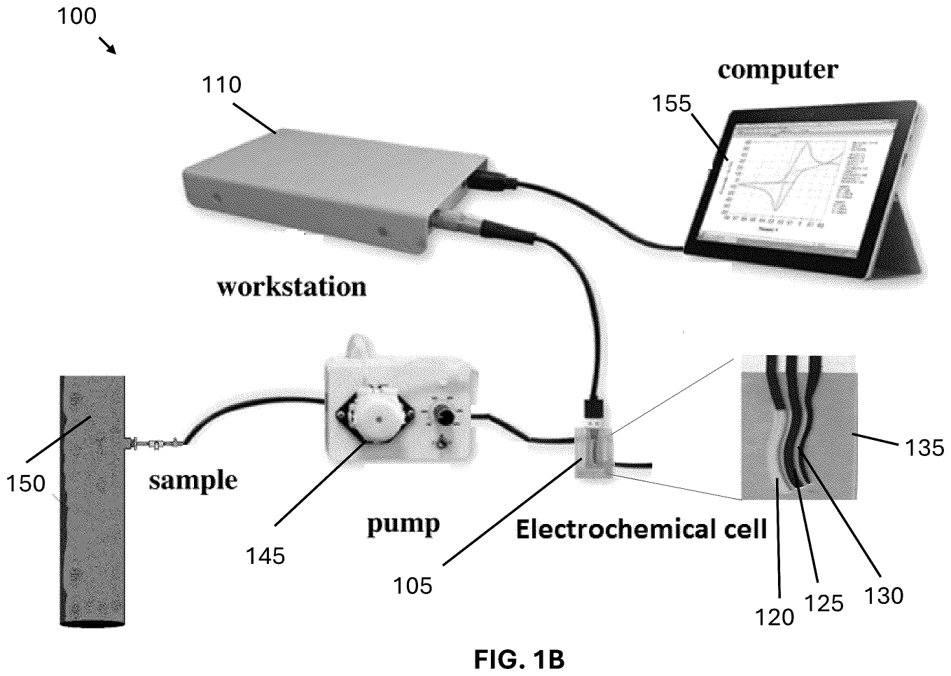

In one or more embodiments, steps of the present detection method are performed using a functionalized fabric sensor as a working electrode and a voltammetry system (e.g., cyclic voltammetry, linear sweep voltammetry) for performing electrochemical measurements. Exemplary voltammetry system configurations are shown in FIGS. 1A-1B in accordance with one or more embodiments. In at least one embodiment, as exemplified in FIG. 1A, the voltammetry system 100 can include an electrochemical cell 105 operatively connected to a potentiostat 110. The electrochemical cell 105 can comprise a container 115 (e.g., glass container) that includes the functionalized fabric sensor 120 as the working electrode, a reference electrode 125, and a counter electrode 130. A portion of each of the functionalized fabric sensor 120 (working electrode), the reference electrode 125, and the counter electrode 130 are in contact with an electrolyte solution 135 in the container 115. The electrolyte solution 135 can comprise the water sample in which the detection of the corrosion inhibitor is sought. The connections 140 of the three electrodes operatively connects the three electrodes to the potentiostat 110. The potentiostat 110 can be used to control the applied potential of the working electrode (functionalized fabric sensor 120) as a function of the potential of the reference electrode 125. The potentiostat 110 can be a part of a standalone device having a monitor or can be hardware that is operatively connected to a computing device that has a monitor, for example.

FIG. 1B shows an example implementation of the voltammetry system 100 and how it can be operatively connected to a water system in accordance with one or more embodiments. As exemplified in FIG. 1B, in one or more embodiments, the system 100 can further include a pump 145 that is operatively connected to a water system or water sample 150. The pump 145 can be configured to pump a sample of the water from the water system to the electrochemical cell 105 for detection and/or measurement of the corrosion inhibitor in the water sample. As exemplified in FIG. 1B, in one or more embodiments the potentiostat 110 can be in the form of a workstation operatively connected to the electrochemical cell and a computing device 155.

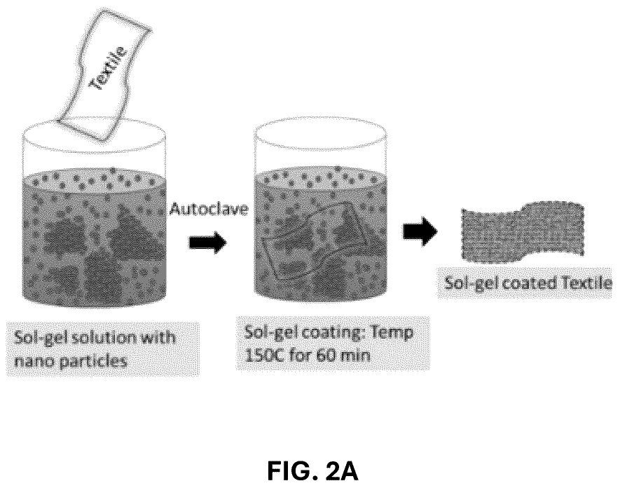

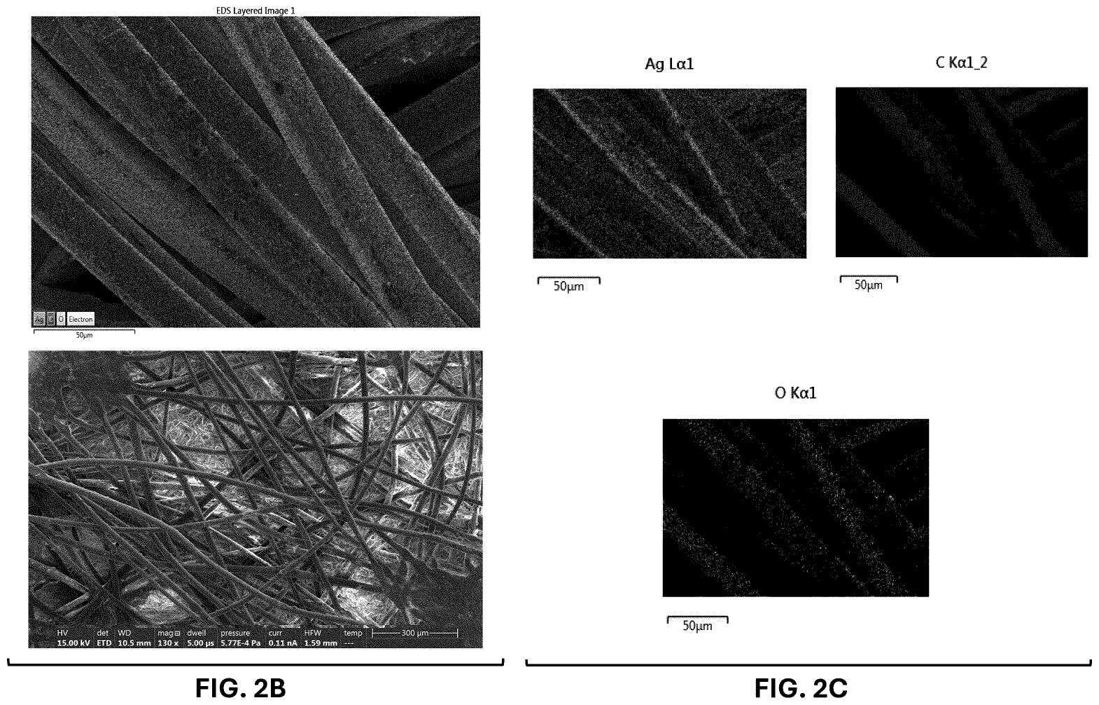

In accordance with one or more embodiments, the functionalized fabric sensor 120 (working electrode) can be fabricated using a sol-gel coating method to attach the electrode metal particles to a textile fabric. An exemplary method for functionalizing a fabric electrode is shown in FIG. 2A and described below.

First, in accordance with one or more embodiments, rice husk waste is cleaned with distilled water and incinerated controllably to form high-purity silica powder at approximately 700° C. for approximately 3 hours. The silica powder is then treated with 1.0M HNO₃ for approximately 24 hours, filtered and washed with deionized water until a constant pH value to form rice husk ash and dried at approximately 150° C. Approximately 10.0 g of rice husk ash is then added to approximately 250 ml of 6.0 M NaOH, stirred for approximately 12 hours and filtered to remove undissolved material. The filtrate is then rapidly titrated with approximately 10% of one or more metal solutions prepared separately by dissolving the metal in approximately 200 ml of 3.0 M HNO₃. In one or more embodiments, the one or more metal solutions comprise silver (Ag). In at least one embodiments, the one or more metal solutions comprise Ag, zinc (Zn), selenium (Se), iron (Fe), copper (Cu), or zirconium (Zr), or combinations thereof. In one or more embodiments, the metal solution(s) are added to the filtrate drop by drop, and titration follows until an approximate pH of 5 is reached in the titrate, thereby forming a sol-gel solution containing metal nanoparticles (e.g., Ag nanoparticles). In such embodiments, a pH of 5 is the minimum (threshold) pH for gel formation—at a pH below 5, the gel will not form (it exists as a solution), and at a pH above 5, a precipitate is formed.

Next, textile fabrics (e.g., non-woven textiles, cotton, polyester, and/or blended textiles) are then added to the sol-gel solution containing metal nanoparticles as shown in FIG. 2A. In one or more embodiments the textile fabrics can be precleaned. The textile fabrics are immersed in the sol-gel solution and then autoclaved at approximately 150° C. for approximately 1 hour to form surface-modified coated textile cloths. Finally, the surface-modified coated cloths are then removed from the sol-gel solution, washed with water, and then dried at room temperature to form the functionalized fabric sensor.

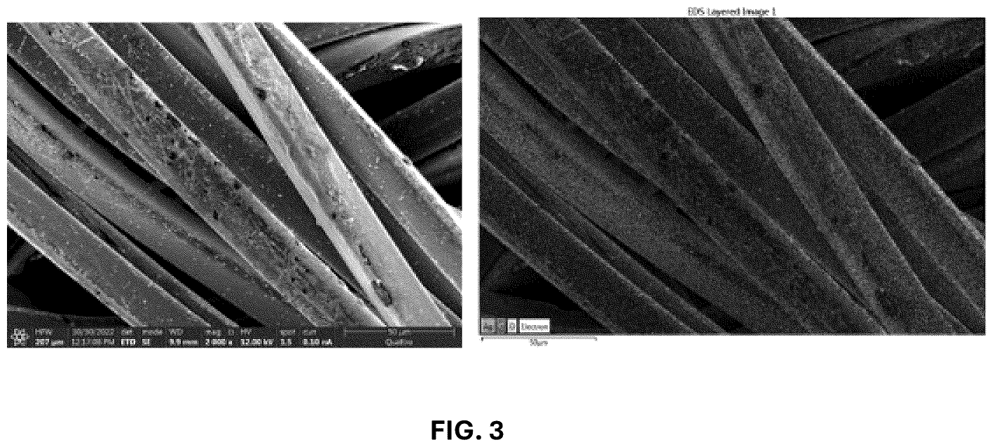

Scanning electron microscopy (SEM), energy dispersive x-ray spectrometry (EDX), and other methods can be used to characterize the chemical properties (e.g., elemental distribution) of the textile fabric and the resulting functionalized fabric sensor. FIG. 2B displays energy dispersive x-ray spectroscopy (EDX) images of a cotton fabric functionalized with Ag nanoparticles with elemental distribution (top image), and a regular scanning electron microscopy (SEM) image of the cotton fabric functionalized with Ag nanoparticles in accordance with one or more embodiments. FIG. 2C displays energy dispersive x-ray spectrometry (EDX) elemental map of Ag, C, and O, in the cotton fabric electrode in accordance with one or more embodiments. Similarly, FIG. 3 displays and SEM image (left panel) and EDX elemental image (right panel) of a functionalized silver fabric electrode in accordance with one or more embodiments.

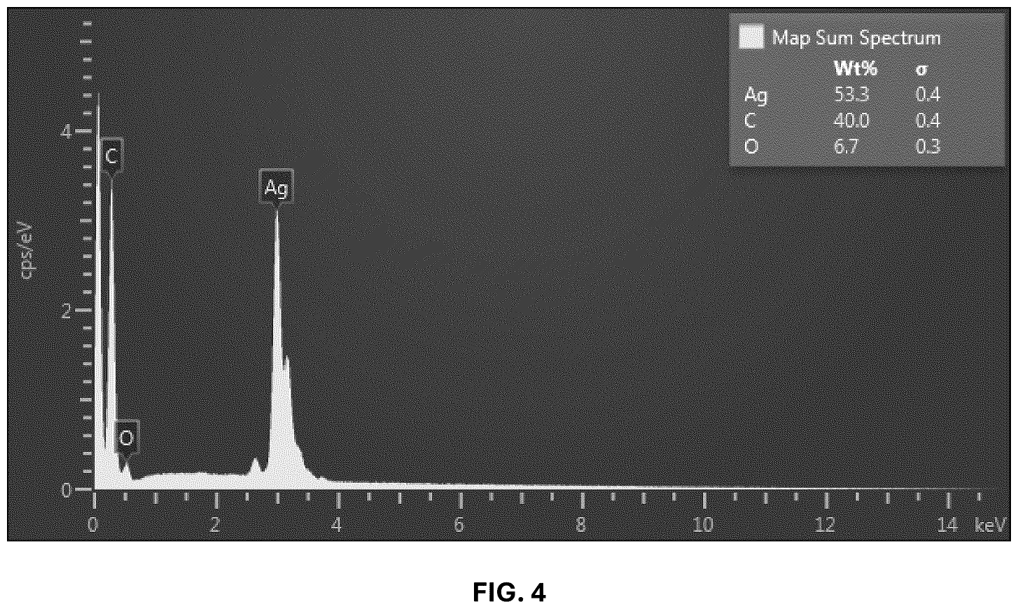

FIG. 4 shows an EDX elemental map of a functionalized silver fabric electrode in accordance with one or more embodiments. As exemplified in the elemental map of FIG. 4, the functionalized silver fabric electrode is composed of a carbon base with a coating of silver (Ag) functionalities.

In accordance with one or more embodiments, as mentioned above, the textile fabrics are functionalized with Ag (e.g., Ag nanoparticles) in the sol-gel solution. Ag nanoparticles (AgNPs) coated fabric electrode exhibits enhancing sensitivity for detecting corrosion inhibitors. AgNP-coated fabric has several unique properties, such as an exceptionally high surface-to-volume ratio, meaning a more significant amount of reactive surface area is available for interaction with target molecules (e.g., nitrogen compounds ions). This characteristic ensures that even minimal concentrations of an analyte can be detected, as there are more collision opportunities between the analyte and the nanoparticle surface. Additionally, AgNPs facilitate higher electron transfer at the electrode surface. When AgNPs are used to modify an electrode, they create a conductive network that promotes electron transfer during electrochemical reactions. This enhanced conductivity leads to stronger signals (currents) for a given concentration of analyte, thereby improving the sensitivity of the detection for the functionalized silver fabric electrode. Additionally, AgNPs can also improve specific interactions with the target analyte (e.g., nitrogen compounds). This specificity reduces background interference and enhances the selectivity and sensitivity of the detection method.

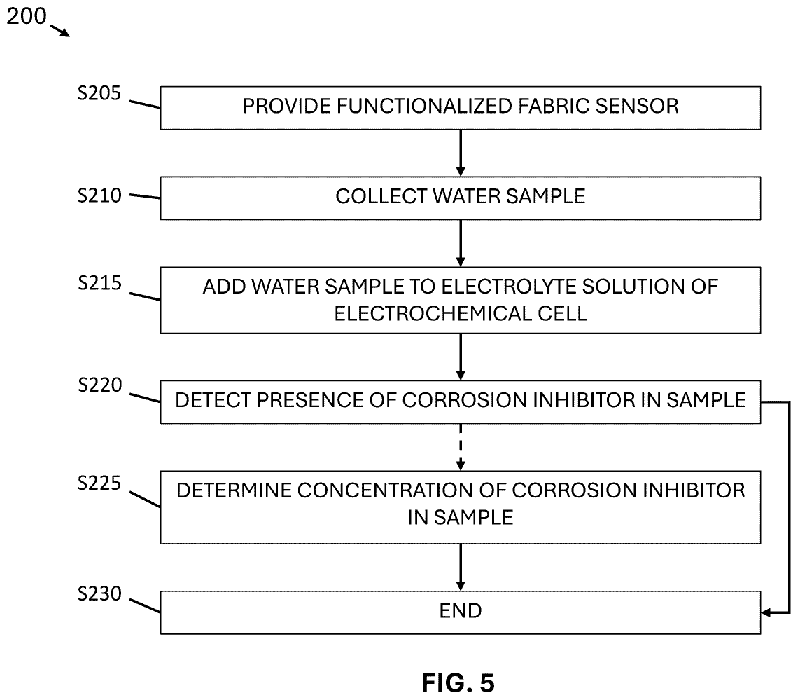

FIG. 5 displays a flow diagram of an exemplary method 200 for electrochemical detection of residual corrosion inhibitor in a water system in accordance with one or more embodiments.

With reference to FIG. 5, the method 200 begins at step S205, where a functionalized fabric sensor is provided. In one or more embodiments, the functionalized fabric sensor can be made in a manner as described in the fabrication method above or in a similar method. In alternative embodiments, other fabrication methods can be used to functionalize the fabric, such as drop casting, spray coating, etc. However, the sol-gel coating method as described in the present application is advantageous as it provides a robust and stable electrode. As described above, in one or more embodiments, the functionalized fabric sensor comprises a textile fabric having a surface coating. In one or more embodiments, the textile fabric can be a non-woven textile, cotton, polyester, or a blended textile. In at least one embodiment, the textile fabric is precleaned. The surface coating comprises a sol-gel solution and metal particles, and the textile fabric is functionalized by the metal particles to form a working electrode. In one or more embodiments, the metal particles are silver nanoparticles. As mentioned above, the silver nanoparticles modify the working electrode to improve conductivity and enhance detection of a corrosion inhibitor. In other embodiments, the metal particles comprise zinc (Zn), selenium (Se), iron (Fe), copper (Cu), or zirconium (Zr), silver (Ag), or combinations thereof.

With continued reference to FIG. 5, at step S210, a water sample is collected that is suspected of comprising a corrosion inhibitor. In one or more embodiments, the water sample can be taken from a reservoir or a well (e.g., water supply well for maintaining reservoir pressure) or other water source. In one or more embodiments, the water sample can be collected via a conduit that is operatively connected to a pump such that a water sample is pulled from the water source via the pump. In one or more embodiments, the corrosion inhibitor that is in or suspected of being in the water sample is a nitrogen-based corrosion inhibitor. For instance, the nitrogen-based corrosion inhibitor can be a quaternary amine, an imidazoline, an amide, or combinations thereof.

At step S215, the water sample is added to an electrolyte solution in an electrochemical cell. In one or more embodiments, the water sample is transferred from a water source to the electrochemical cell via a pump. In one or more embodiments, the amount of water transferred to the electrochemical cell can be between approximately 1-10 mL; however, the specific amount is dependent on the size of the electrochemical cell. In one or more embodiments, the water sample is 1 part to 9 parts of the electrolyte solution, giving a 10% of water sample in the final solution being measured in the electrochemical cell. As referenced above, the electrochemical cell includes the functionalized fabric sensor as the working electrode, a reference electrode, and a counter electrode, and a portion of the electrodes are in contact with the electrolyte solution. In one or more embodiments, the electrolyte solution comprises a potassium compound. For instance, in one or more embodiments the potassium compound is KCl, or K₃Fe(CN)₆ (potassium hexacyanoferrate (III)). In one or more embodiments, the electrolyte solution can also include comprises a buffer solution, such as phosphate-buffered saline (PBS).

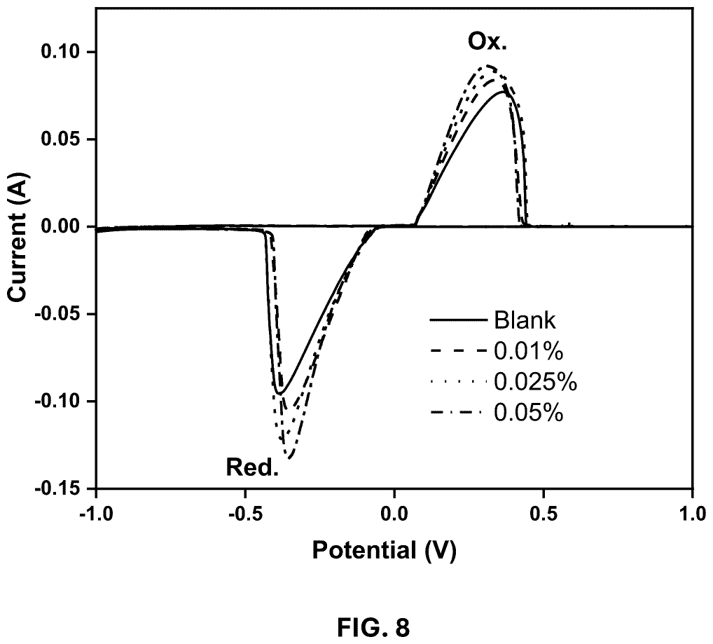

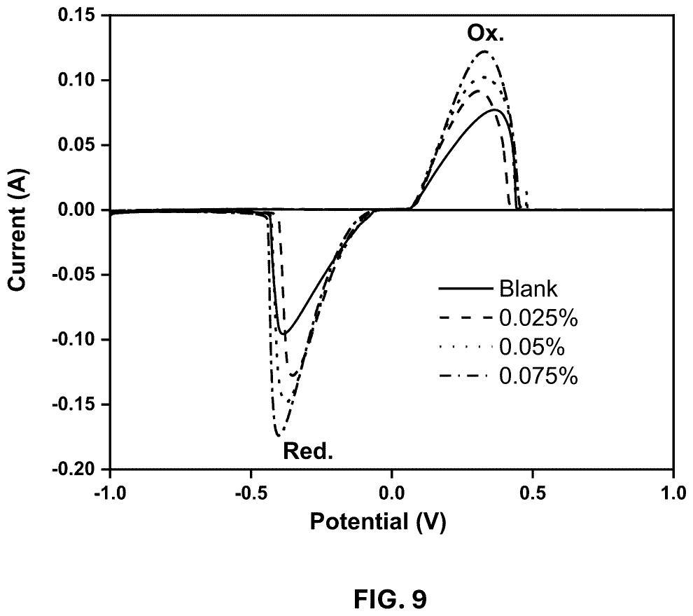

At step S220, the presence (or absence) of the corrosion inhibitor is detected in the water sample in the electrolyte solution of the electrochemical cell. Specifically, the electrodes of the electrochemical cell are operatively connected to a potentiostat and the electrochemical cell is utilized at a given or predetermined potential to produce a measurable current signal to detect the presence of the corrosion inhibitor. For example, in one or more embodiments, the corrosion inhibitor sample has a potential of approximately +/−0.4 V. In such an embodiment, there is a measurement window of approximately −1 to +1 V, which covers the specific potential of the corrosion inhibitor, as exemplified in FIGS. 8 and 9. If the corrosion inhibitor is present, the peaks will show up approximately +/−0.4 V. If absent, no such peaks will be seen. The given potential refers to the potential of the working electrode relative to the reference electrode. So, if one changes the working electrode, the “given potential” will change. The range of potentials for nitrogen-based corrosion inhibitors is preferably −1 to +1 V. In one or more embodiments, a cyclic voltammetry technique or a linear sweep voltammetry technique is utilized for detecting the corrosion inhibitor in the sample using the electrochemical cell and the potentiostat.

Optionally, at step S225, a concentration of the corrosion inhibitor in the water sample can be determined. In one or more embodiments, the determination of the concentration of the corrosion inhibitor in the water sample can be made based on the voltammetry results (e.g., cyclic voltammetry technique or a linear sweep voltammetry results), and more specifically, based on the measurable current signal produced by the electrochemical cell. For example, in one or more embodiments, The reduction peaks of the cyclic voltammograms for standard solutions for each corrosion inhibitor are used to establish calibration curves (current vs. concentration). Then, the concentration of the corrosion inhibitor in the sample can be determined from the calibration curve based on the measured reduction peaks of the cyclic voltammograms of the sample.

At step S230, the method ends.

The present methods for detecting corrosion inhibitors in water samples provide enhanced sensitivity for detecting residual amounts of corrosion inhibitors, and are more cost effective than conventional methods. For example, mass spectrometry is conventionally used for detection of corrosion inhibitors, but these mass spectrometry systems impose a huge cost burden. In contrast, the functionalized fabric sensor system of the present application includes inexpensive tools to monitor redox species and furnish good selectivity for those species based on the selected oxidation-reduction potential. Moreover, the diversity in the selection and modification of electrode materials provide a quick adjustment, rather than switching of the sensor functions against a certain analyte or a class of analytes. Accordingly, the present system and method uses cheap instrumentation connected to novel functionalized fabric electrodes that can be modified to detect different types of corrosion inhibitors (e.g., nitrogen-based corrosion inhibitors), and furthermore, multi-array probes can be designed to detect many analytes. Multi-array probes can detect different elements based on the applied voltage which produces specific reduction current. For example, multiple analytes can be detected by doing the measurement in a different mode, such as stripping voltammetry, instead of cyclic voltammetry. In stripping voltammetry, the potential is first scanned negatively over a wide range (e.g., 0 to −2) to deposit all analytes, then the potential scan is reversed from −2 to 0 to strip individual analytes from the electrode, and as each of them comes off at its specific potential, a peak will develop for that specific analyte.

The above aspects and other aspects of the methods of the present application can be further understood through the following examples.

### Example 1

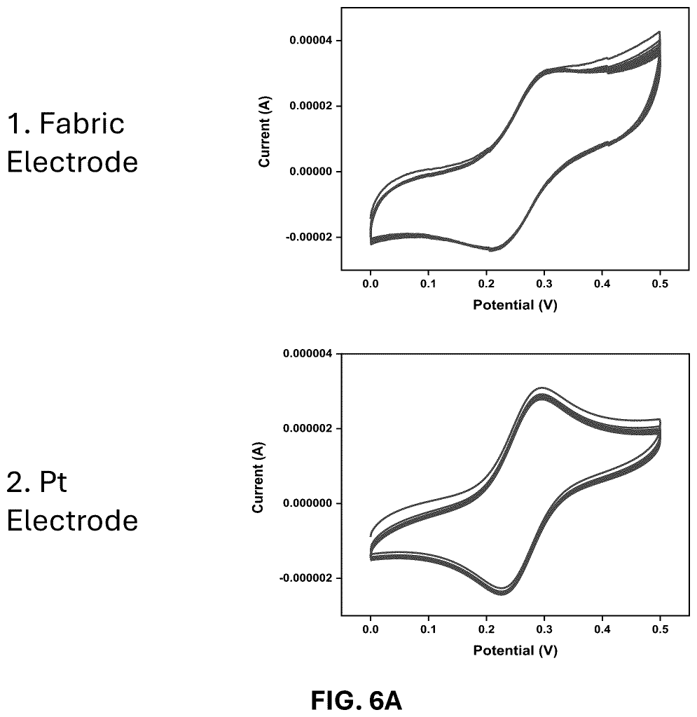

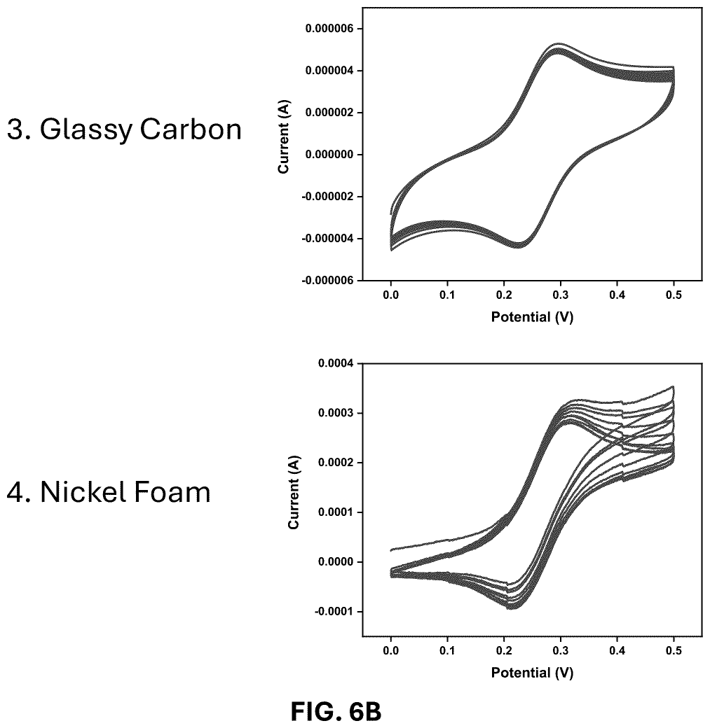

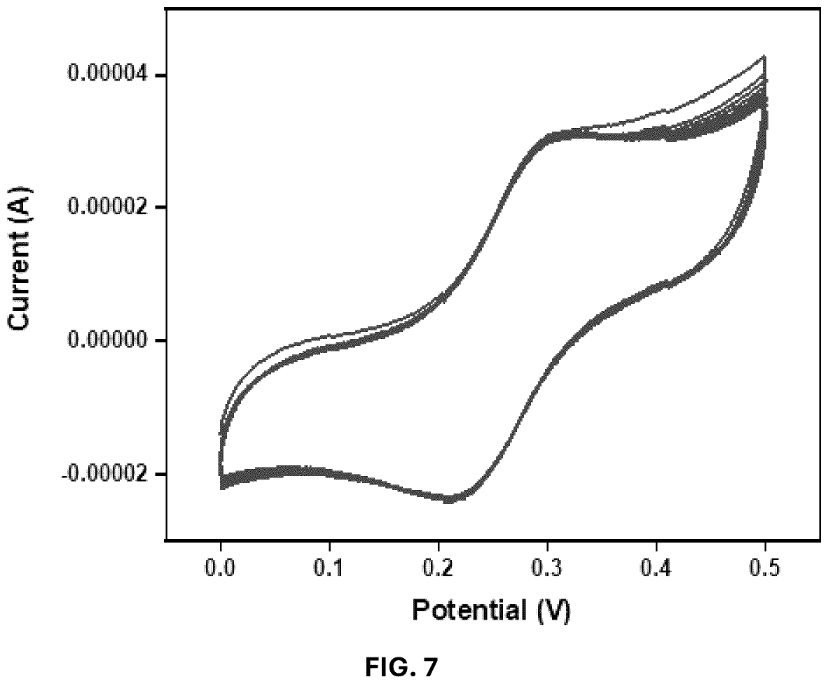

Several electrochemical measurements using an electrochemical cell and a potentiostat were conducted in the presence of two commercial water-soluble corrosion inhibitors that are based on pyridinium quaternary ammonium salt. The experiments utilized a functionalized fabric electrode in accordance with one or more embodiments herein, as well as other working electrodes, including a glass carbon graphite electrode, a platinum (Pt) electrode, and a graphite sheet and nickel foam electrode. Specifically, the functionalized fabric electrode was comprised of cotton fabric and silver. The experiments were carried out to verify the electrochemical activity of the various working electrodes under given experimental conditions and their suitability for detecting corrosion inhibitor residuals in an aqueous (electrolyte) solution. Measurements were made at room temperature, using distilled water, and a series of standard samples with known salt and buffer concentrations. The experiments were carried out in the electrolyte solutions: 0.5 mM solution of potassium hexacyanoferrate (III) (K₃Fe(CN)₆) using cyclic voltammetry (CV) technique, in order to determine the electrochemical activity of the functionalized fabric electrode. The CV results using the K₃Fe(CN)₆ are shown in FIGS. 6A-6B and FIG. 7, with the CV results for the functionalized fabric electrode shown in FIG. 7. The CV results in FIG. 7 showed clear oxidation and reduction reaction peaks, with the oxidation peak around 0.30 V and a reduction peaks around 0.22 V. These results indicate that the functionalized fabric electrode has good electrochemical activity.

### Example 2

Concentration-dependent measurements were carried out using two commercial corrosion inhibitors in the low concentration range of 0.0-0.075%. The two commercial water-soluble corrosion inhibitors are based on pyridinium quaternary ammonium salt. The CV results for these concentration-dependent measurements are presented in FIGS. 8 and 9. FIG. 8 displays CV results of various concentrations of the first commercial corrosion inhibitor (0.01%, 0.025%, 0.05%) in water with the fabric electrode in the presence of a KCl supporting electrolyte and PBS buffer at pH 7 at room temperature. FIG. 9 displays CV results of various concentrations (0.025%, 0.050%, 0.075%) of the second commercial corrosion inhibitor in water with the fabric electrode in the presence of a KCl supporting electrolyte and PBS buffer at pH 7 at room temperature. As shown in results of FIGS. 8 and 9, there is a subtle shift in the oxidation and reduction potentials as the concentration changes. These subtle shifts may be attributed to anomalous behavior of the fabric electrode when used to measure the CV of whereby it did not give any oxidation or reduction peaks until after about 10 cycles.

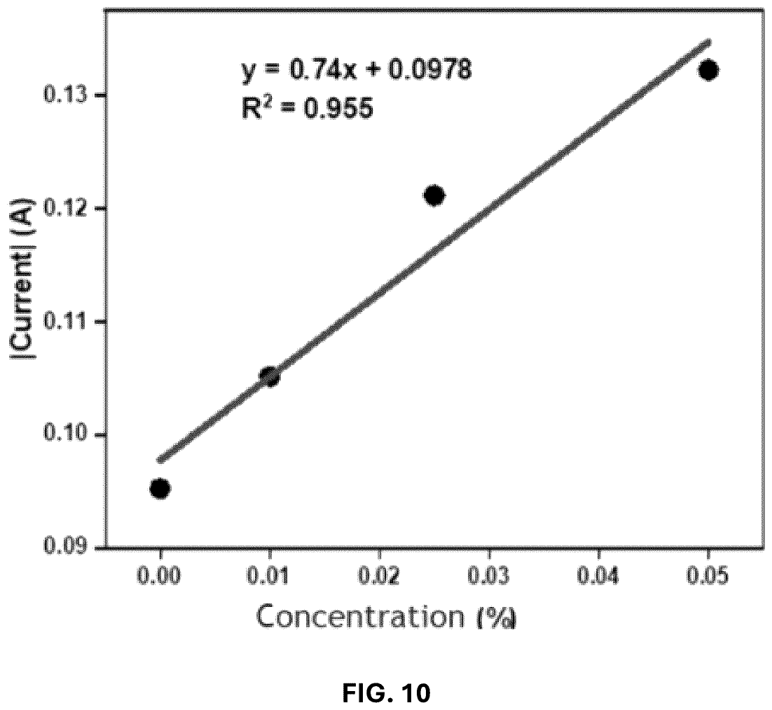

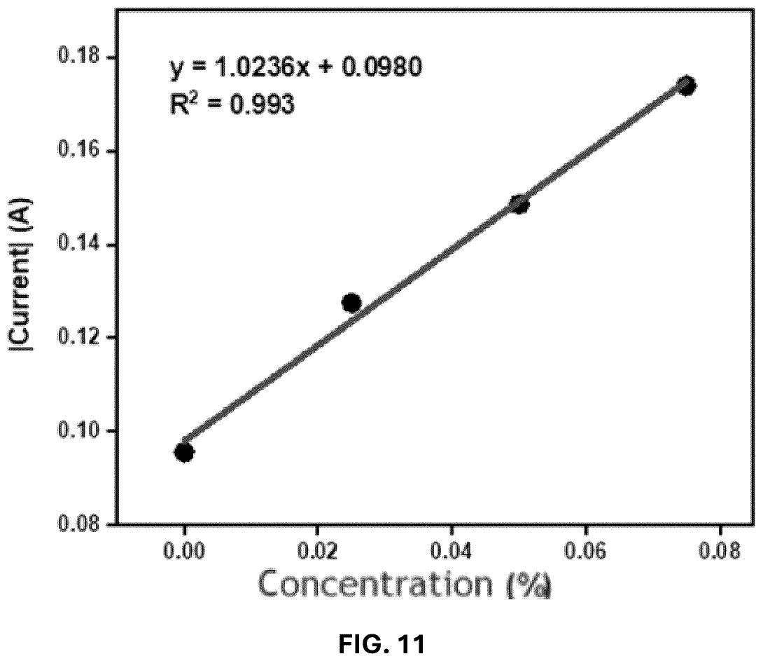

The CV generated data of FIGS. 8 and 9 were then used for plotting calibration graphs, which are shown in FIGS. 10 and 11. FIG. 10 shows a linear calibration curve for the first commercial inhibitor sample in the concentration range of 0.0 to 0.05%. FIG. 11 shows a linear calibration curve for the second commercial inhibitor sample in the concentration range of 0.0 to 0.075%. The reduction peaks of the cyclic voltammograms in FIGS. 8 and 9 were used for the calibration curves. It is noted that the fabric electrode worked with the corrosion inhibitor samples in these experiments.

### Example 3

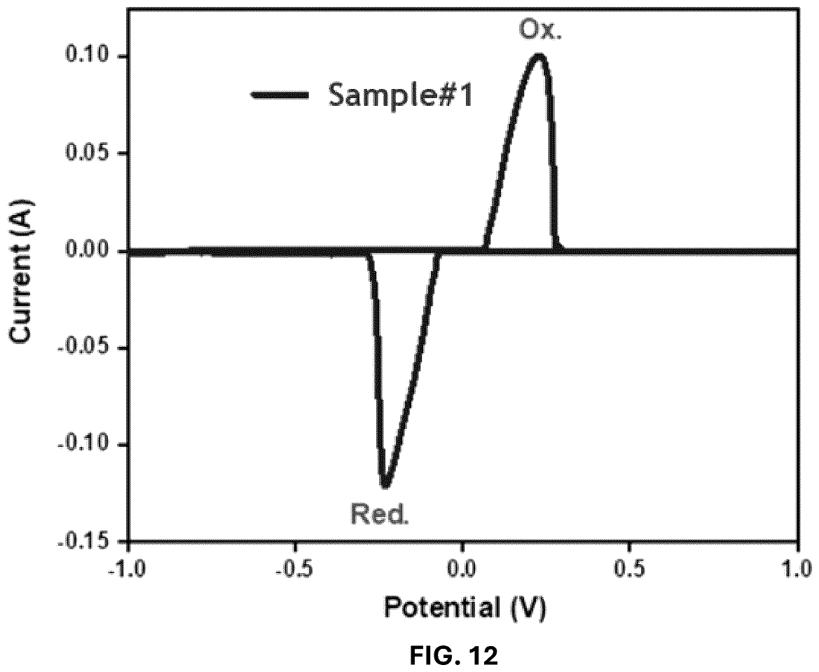

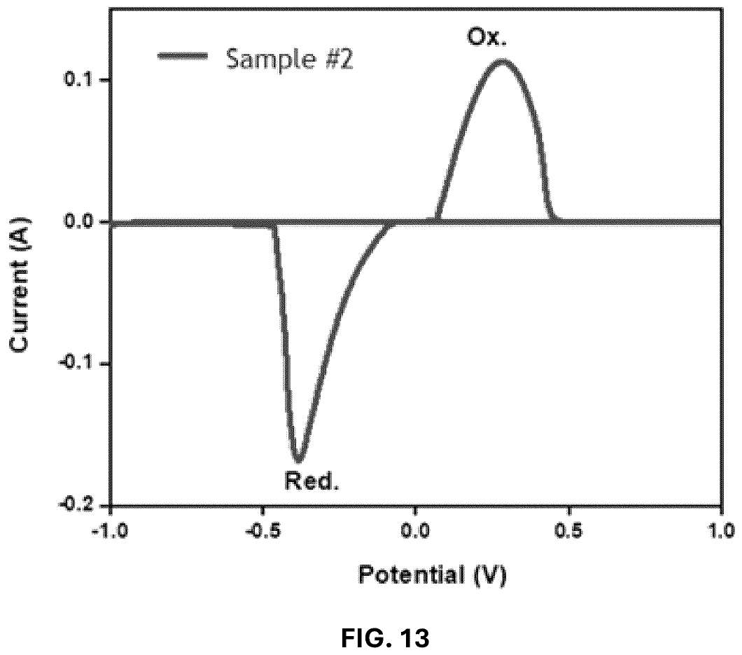

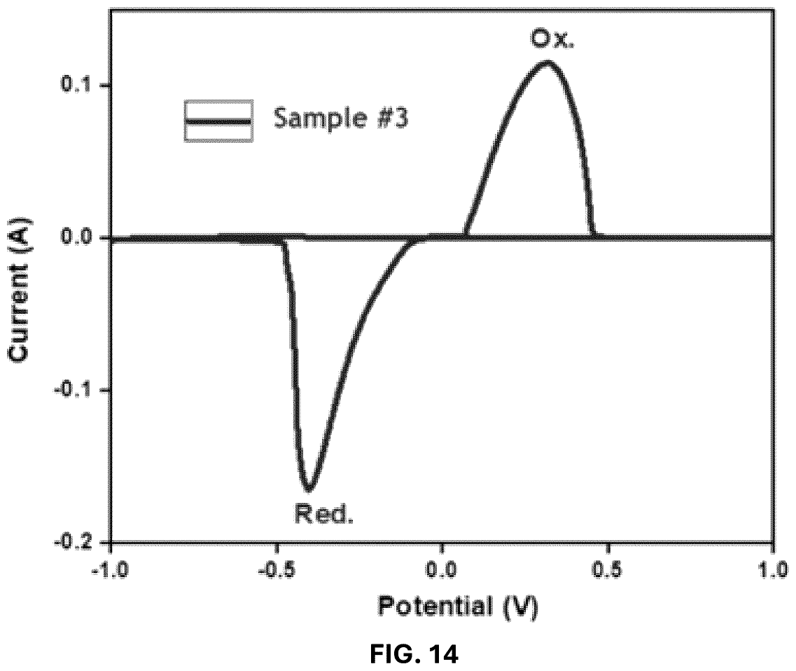

Three field water treated samples were collected and analyzed using the developed fabric electrode and CV technique. The obtained cyclic voltammograms for the three analyzed samples (samples 1-3) are illustrated in FIGS. 12-14. In each case, 1 mL of the sample was added to the buffered electrolyte solution, giving a final volume of 10 mL. This indicates 10 times dilution. The analyzed corrosion inhibitor-treated water samples showed very good cyclic voltammograms, which allowed for the estimation of the concentration of the corrosion inhibitor from the established calibration curve. The estimated concentration of corrosion inhibitor in each treated water sample after accounting for 10× dilution is tabulated in Table 1.

TABLE 1
Calculated Concentration

| Sample #1 | Sample #2 | Sample #3 |
|---|---|---|
| 0.226 | 0.694 | 0.662 |

It is to be understood that not all components and/or steps described and illustrated with reference to the figures are required for all embodiments or arrangements. Further, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “including,” “comprising,” or “having,” “containing,” “involving,” and variations thereof herein, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Notably, the figures and examples above are not meant to limit the scope of the present disclosure to a single implementation, as other implementations are possible by way of interchange of some or all the described or illustrated elements. Moreover, where certain elements of the present disclosure can be partially or fully implemented using known components, only those portions of such known components that are necessary for an understanding of the present disclosure are described, and detailed descriptions of other portions of such known components are omitted so as not to obscure the disclosure. In the present specification, an implementation showing a singular component should not necessarily be limited to other implementations including a plurality of the same component, and vice versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). It is to be understood that dimensions discussed or shown are drawings according to one example and other dimensions can be used without departing from the disclosure.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

What is claimed is:

1. A method for electrochemical detection of residual corrosion inhibitor in a water system, the method comprising:
providing a functionalized fabric sensor, wherein the functionalized fabric sensor comprises a textile fabric having a surface coating comprising metal particles, and wherein the textile fabric is functionalized by the metal particles to form a working electrode;
collecting a water sample suspected of comprising a corrosion inhibitor, wherein the corrosion inhibitor is a nitrogen-based corrosion inhibitor;
adding the water sample to an electrolyte solution in an electrochemical cell; and
detecting the presence of the corrosion inhibitor in the water sample in the electrolyte solution of the electrochemical cell,
wherein the electrochemical cell includes the functionalized fabric sensor as the working electrode, a reference electrode, and a counter electrode, and the electrodes are operatively connected to a potentiostat, and wherein the electrochemical cell is utilized at a predetermined potential to produce a measurable current signal to detect the presence of the corrosion inhibitor.

2. The method of claim 1, wherein the metal particles comprise silver nanoparticles, and wherein the silver nanoparticles modify the working electrode to improve conductivity and enhance detection of the corrosion inhibitor.

3. The method of claim 1, wherein the surface coating comprises a sol-gel solution.

4. The method of claim 1, wherein the electrochemical cell utilizes a cyclic voltammetry technique or a linear sweep voltammetry technique.

5. The method of claim 1, wherein the corrosion inhibitor is a nitrogen-based corrosion inhibitor.

6. The method of claim 5, wherein the nitrogen-based corrosion inhibitor is a quaternary amine, an imidazoline, an amide, or combinations thereof.

7. The method of claim 1, wherein the electrolyte solution comprises a potassium compound.

8. The method of claim 7, wherein the potassium compound is KCl, or K₃Fe(CN)₆ (potassium hexacyanoferrate (III)).

9. The method of claim 7, wherein the electrolyte solution further comprises a buffer solution.

10. The method of claim 9, wherein the buffer solution comprises phosphate-buffered saline (PBS).

11. The method of claim 1, further comprising:
determining a concentration of the corrosion inhibitor in the water sample based on voltammetric measurements of the system.

12. The method of claim 1, wherein the textile fabric is a non-woven textile, cotton, polyester, or a blended textile.

13. The method of claim 1, wherein the textile fabric is precleaned.

14. The method of claim 1, wherein the corrosion inhibitor has a concentration in the water sample in the range of approximately 0.01-0.075%.

15. The method of claim 1, wherein the water sample is added to the electrolyte solution at a ratio of 1 part water sample to 9 parts electrolyte solution.
